(19) United States
(12) Patent Application Publication
(10) Pub. No.: US 2018/0164257 A1
(43) Pub. Date: Jun. 14, 2018

(54) ULTRASOUND PROBE UNIT AND ULTRASOUND DIAGNOSTIC APPARATUS

(71) Applicant: Konica Minolta, Inc., Chiyoda-ku Tokyo (JP)

(72) Inventors: Takehiko SUGINOUCHI, Tokyo (JP); Hideo HONGO, Kanagawa (JP)

(21) Appl. No.: 15/840,731

(22) Filed: Dec. 13, 2017

(30) Foreign Application Priority Data
Dec. 13, 2016 (JP) 2016-241491

(51) Int. Cl. G01N 29/26; G01N 29/34; A61B 8/00; A61B 8/14

(52) U.S. Cl. CPC G01N 29/262; G01N 29/348; A61B 8/54; A61B 8/4488; A61B 8/145; A61B 8/4461

(57) ABSTRACT

The driving circuit includes a variable-output switch-mode power supply; a power amplifier into which an output voltage of the variable-output switch-mode power supply is input, and which outputs a driving voltage to an actuator on the basis of the output voltage; and a comparator that compares a first target voltage based on the driving voltage of the actuator with a second target voltage based on the output voltage of the variable-output switch-mode power supply, in which when an absolute value of the second target voltage is smaller than an absolute value of the first target voltage as a result of the comparison in the comparator, the control circuit performs switching control of the variable-output switch-mode power supply so as to increase the absolute value of the second target voltage to the absolute value of the first target voltage or larger.

### CROSS REFERENCE TO RELATED APPLICATIONS

The present invention claims priority under 35 U.S.C. § 119 to Japanese Patent Application 2016-241491 filed on Dec. 13, 2016, the entire contents of which is incorporated herein by reference.

### BACKGROUND

### Technological Field

The present invention relates to an ultrasound probe unit of an ultrasound diagnostic apparatus that utilizes ultrasound, and an ultrasound diagnostic apparatus.

### Description of Related Art

Ultrasound diagnostic apparatuses that irradiate the inside of test objects, and receive and analyze the reflected waves to inspect the inside of the test objects have been widely used. Such ultrasound diagnostic apparatuses can examine test objects non-destructively and non-invasively, and thus are widely employed in various applications, such as a medical-purpose inspection and an inspection of the inside of architectural constructions.

In an ultrasound diagnostic apparatus, a plurality of acoustic elements (transducers) that convert a voltage signal into ultrasonic vibration and vice versa are arranged in a predetermined direction (scanning direction), and the acoustic elements emit ultrasound upon application of a driving voltage. Such an ultrasound diagnostic apparatus can obtain two-dimensional data in nearly real-time by switching (scanning), over time, acoustic elements that detect voltage changes due to incidence of reflected ultrasound.

There exists a technique for obtaining three-dimensional images in nearly real-time by moving arranged acoustic elements back and forth (rocking) perpendicularly to the scanning direction on the emission/incident plane of ultrasound. By obtaining three-dimensional images using such a technique, operators can more easily know three-dimensional shapes and/or positional relationships of test objects, which are difficult to perceive in two-dimensional images.

An example of an ultrasound probe (probe) that rocks arranged acoustic elements perpendicularly to the scanning direction is described in Japanese Patent Application Laid-Open No. 2004-016750, for example. FIG. 1 shows an example configuration of conventional ultrasound probe unit 800 as described in Japanese Patent Application Laid-Open No. 2004-016750. As shown in FIG. 1, ultrasound probe unit 800 includes ultrasound probe 810, and connector housing 820, which is a connector that connects ultrasound probe 810 to an ultrasound diagnostic apparatus (not shown).

As shown in FIG. 1, ultrasound probe 810 that enables three-dimensional scanning includes acoustic element array 811 composed of a plurality of acoustic elements, and rocking mechanism 812 that mechanically rocks the acoustic element array for scanning. Rocking mechanism 812 may include an encoder that detects the scanning position of ultrasound probe 810. As shown in FIG. 1, connector housing 820 includes driving circuit 821 that controls a stepping motor of rocking mechanism 812, and control circuit 822 that controls driving circuit 821.

Conventional ultrasound probe unit 800 having the above configuration performs three-dimensional scanning by controlling rocking mechanism 812 of ultrasound probe 810 by driving circuit 821 installed in connector housing 820 under the control by control circuit 822 on the basis of a rocking command signal from an ultrasound diagnostic apparatus. In this configuration, since driving circuit 821 is provided in connector housing 820, rocking control of acoustic element array 811 can be performed on the side of ultrasound probe unit 800, not on the side of an ultrasound diagnostic apparatus body. This enables rocking control of acoustic element array 811 without any trouble even when, for example, ultrasound probe unit 800 is used or installed in various ultrasound diagnostic apparatus bodies.

Commonly, in other words, in cases other than ultrasound diagnostic apparatuses, highly efficient switching amplifiers (class-D amplifiers) are often used in driving circuits of various motors, such as a stepping motor, and a three-phase DC motor or AC motor. A switching amplifier is a digital amplifier that performs amplification by switching operation using pulses in pulse width modulation (PWM), for example.

When a class-D amplifier is used in a driving circuit for a stepping motor of an ultrasound diagnostic apparatus, however, a harmonic is generated by PWM pulse signals in some cases. As described above, since driving circuit 821 is provided inside connector housing 820, which is a connection section with the ultrasound diagnostic apparatus body, there is a risk that a harmonic generated by a class-D amplifier may be superimposed on ultrasonic reception signals generated by ultrasound probe 810. Under such a situation, when ultrasound images are generated by performing image processing on the side of the ultrasound diagnostic apparatus body on the basis of the harmonic-superimposed ultrasound reception signals, accurate diagnosis becomes difficult due to harmonic components emerging on images as noise. In order to avoid this situation, a linear amplifier (class-AB amplifier, for example) is preferably used as a driving circuit for a rocking mechanism (stepping motor) of the ultrasound diagnostic apparatus.

As ultrasound diagnostic apparatuses, in addition to stationary-type apparatuses with relatively large body sizes, hand-carry apparatuses of laptop and portable types, for example, are widely used. In such hand-carry apparatuses, the body sizes become relatively small to ensure portability, and accordingly downsizing of connector housings is needed.

In general, linear amplifiers generate more heat than switching amplifiers. Heat generated by a driving circuit having an amplifier is released from a connector housing to the neighboring area. When a connector housing is downsized, however, an area (surface area of the connector housing), through which heat generated by a driving circuit is released, becomes small. Accordingly, there is a risk that the housing temperature may become higher than in the case of a large connector housing. In view of this and safety, prevention of the temperature rise in a connector housing is needed.

### SUMMARY

An object of the present invention is to provide an ultrasound probe unit and an ultrasound diagnostic apparatus that reduce heat generation by a circuit inside a connector housing.

In order to achieve at least one of the abovementioned objects, according to an aspect of the present invention, an ultrasound probe unit reflecting one aspect of the present invention comprises: an ultrasound probe including an acoustic element array and a rocking mechanism having an actuator that moves the acoustic element array in a direction crossing a scanning direction; a driving circuit that drives the actuator; and a control circuit that controls the driving circuit. The driving circuit includes: a variable-output switch-mode power supply; a power amplifier into which an output voltage of the variable-output switch-mode power supply is input, and which outputs a driving voltage to the actuator on the basis of the output voltage; and a comparator that compares a first target voltage based on the driving voltage of the actuator with a second target voltage based on the output voltage of the variable-output switch-mode power supply. When an absolute value of the second target voltage is smaller than an absolute value of the first target voltage as a result of comparison in the comparator, the control circuit performs switching control of the variable-output switch-mode power supply so as to increase the absolute value of the second target voltage to the absolute value of the first target voltage or larger.

An ultrasound diagnostic apparatus reflecting one aspect of the present intention comprises the above-mentioned ultrasound probe unit and an ultrasound diagnostic apparatus body, in which the ultrasound diagnostic apparatus body causes the ultrasound probe to transmit an ultrasonic transmission signal to a test object, and generates an ultrasound image on the basis of an ultrasonic reception signal generated by the ultrasound probe that has received a reflected wave from the test object.

An ultrasound diagnostic apparatus reflecting one aspect of the present intention comprises an ultrasound probe unit and an ultrasound diagnostic apparatus body that causes the ultrasound probe unit to transmit an ultrasonic transmission signal to a test object, and generates an ultrasound image on the basis of an ultrasonic reception signal generated by the ultrasound probe unit that has received a reflected wave from the test object. The ultrasound probe unit includes: an ultrasound probe including an acoustic element array and a rocking mechanism having an actuator that rocks the acoustic element array perpendicularly to a scanning direction; and a connector housing that is connected to the ultrasound probe via a cable, and that is connected with the ultrasound diagnostic apparatus body. The ultrasound diagnostic apparatus body includes a driving circuit that drives the actuator and a control circuit that controls the driving circuit. The driving circuit includes: a variable-output switch-mode power supply; a power amplifier into which an output voltage of the variable-output switch-mode power supply is input, and which outputs a driving voltage to the actuator on the basis of the output voltage; and a comparator that compares a first target voltage based on the driving voltage of the actuator with a second target voltage based on the output voltage of the variable-output switch-mode power supply. When an absolute value of the second target voltage is smaller than an absolute value of the first target voltage as a result of comparison in the comparator, the control circuit performs switching control of the variable-output switch-mode power supply so as to increase the absolute value of the second target voltage to the absolute value of the first target voltage or larger.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 shows an example configuration of a conventional ultrasound probe unit;

FIG. 2 illustrates a configuration of an ultrasound diagnostic apparatus according to an embodiment of the present invention;

FIG. 3 shows a configuration of an ultrasound probe unit according to the embodiment of the present invention;

FIG. 4 illustrates an example structure of a stepping motor of a rocking mechanism;

FIG. 5 illustrates an example configuration of a driving circuit and a control circuit;

FIG. 6A shows a relationship between a driving voltage V_{D} of a stepping motor of the ultrasound probe unit according to the embodiment of the present disclosure, and output voltages Vₒᵤₜ₊ and Vₒᵤₜ₋ of respective variable-output switch-mode power supplies;

FIG. 6B shows a relationship between an input voltage and an output voltage of a linear amplifier when the input voltage into the linear amplifier is not controlled;

FIG. 7 illustrates a circuit configuration of a variable-output switch-mode power supply;

FIG. 8 shows an example signal waveform in PFM control by a control circuit;

FIG. 9 shows an example output waveform of a variable-output switch-mode power supply when a switching frequency is changed in the opposite directions for every cycle of a driving waveform of a stepping motor;

FIG. 10A shows a relationship between the number of rotations (low-speed rotation) of a stepping motor and an output waveform of a variable-output switch-mode power supply;

FIG. 10B shows a relationship between the number of rotations (high-speed rotation) of the stepping motor and an output waveform of the variable-output switch-mode power supply;

FIG. 10C shows an output waveform when switching control of the variable-output switch-mode power supply is performed by a control circuit during high-speed rotation of the stepping motor; and

FIG. 11 illustrates an example configuration of a voltage divider when the voltage divider outputs target voltages (V_{D}+α) and (V_{D}−α) on the basis of a driving voltage V_{D}.

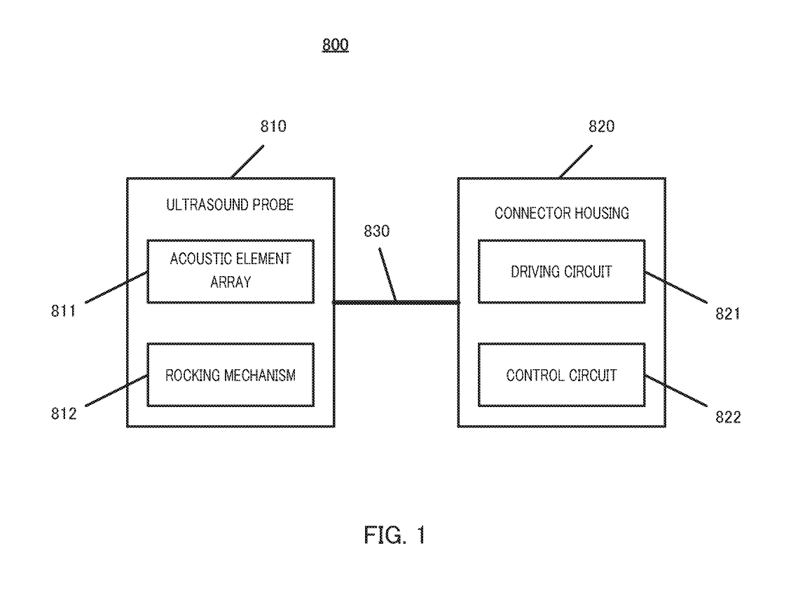

### DETAILED DESCRIPTION OF EMBODIMENTS

Hereinafter, one or more embodiments of the present invention will be described with reference to the drawings. However, the scope of the invention is not limited to the disclosed embodiments.

In the following, the ultrasound probe unit according to the embodiment of the present invention will be described in reference to the drawings. The scope of the invention, however, is not limited to the illustrated examples. In the following, like numerals denote components having like function and configuration, and the description thereof will be omitted.

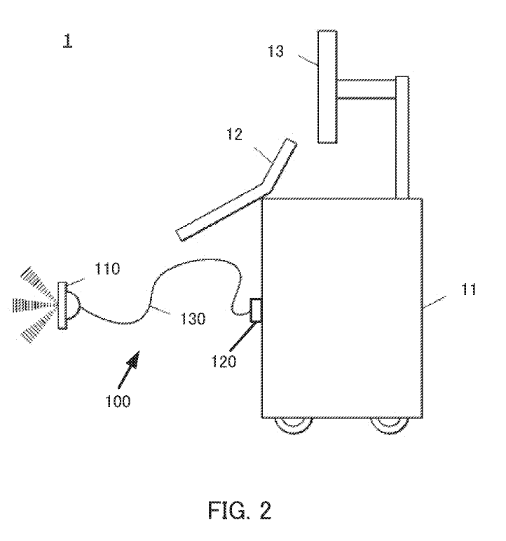

FIG. 2 illustrates a configuration of the ultrasound diagnostic apparatus according to the embodiment of the present invention. As illustrated in FIG. 2, ultrasound diagnostic apparatus 1 includes ultrasound probe unit 100, ultrasound diagnostic apparatus body 11, operation section 12, and display section 13. Ultrasound probe unit 100 includes ultrasound probe 110, connector housing 120, and cable 130.

Ultrasound probe 110 transmits ultrasound (transmission ultrasound) to an test object, such as a living body (not shown), and receives ultrasound reflected inside the test object (reflected ultrasound: echo).

Ultrasound diagnostic apparatus body 11, which is connected to ultrasound probe 110 via cable 130 and connector housing 120, transmits an electrical driving signal to ultrasound probe 110 to cause ultrasound probe 110 to transmit an ultrasound transmission signal to a test object, and generates an ultrasound image of the internal state of the test object on the basis of an ultrasound reception signal generated by ultrasound probe 110 that has received a reflected wave from the inside of the test object. Operation section 12 is an operation device, such as a switch, a button, a keyboard, a mouse, or a touch panel, and receives operations by users of ultrasound diagnostic apparatus 1, such as doctors and technicians. Display section 13 is a display device, such as a liquid crystal display (LCD) or an organic EL display, and shows ultrasound images generated by ultrasound diagnostic apparatus body 11 and/or various screens corresponding to the state of ultrasound diagnostic apparatus 1.

<Configuration of Ultrasound Probe Unit 100>

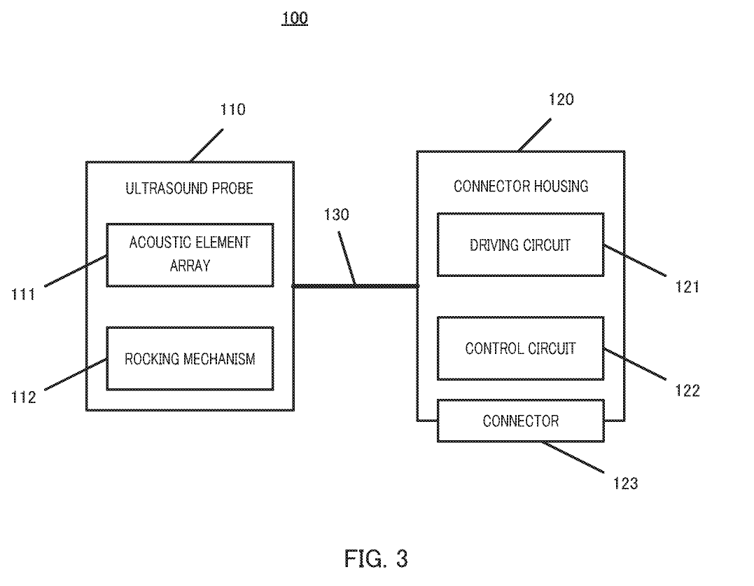

FIG. 3 shows a configuration of ultrasound probe unit 100 according to the embodiment of the present invention. As shown in FIG. 3, ultrasound probe unit 100 includes ultrasound probe 110, connector housing 120, and cable 130. Ultrasound probe unit 100 is connected to the ultrasound diagnostic apparatus body (not shown) via connector housing 120.

Ultrasound probe 110 comes into contact with a test object during ultrasound diagnosis, transmits an ultrasound signal, receives a reflected wave signal, and generates a reception signal. Ultrasound signals are generated on the basis of control signals transmitted from the ultrasound diagnostic apparatus body via connector housing 120 and cable 130. Meanwhile, reception signals received by ultrasound probe 110 are transmitted to the ultrasound diagnostic apparatus body via cable 130 and connector housing 120. Through this process, ultrasound images are generated in the ultrasound diagnostic apparatus body.

As shown in FIG. 3, ultrasound probe 110 includes acoustic element array 111 and rocking mechanism 112. Acoustic element array 111 is composed of acoustic elements, which generate ultrasound by mutual conversion of electrical signals and ultrasound, linearly arranged in the scanning direction, for example. Rocking mechanism 112 is a mechanism for enabling three-dimensional scanning through rocking of acoustic element array 111 to move an ultrasound forming plane. Rocking mechanism 112 is composed of stepping motor 200 as an actuator described hereinafter and a transmission member (not shown), such as a pulley or a belt, for example, and rocks a base (not shown) in which acoustic element array 111 is provided perpendicularly to the scanning direction by the driving force of the stepping motor through the transmission member.

Although not shown in FIG. 3, ultrasound probe 110 may further include, for example, an acoustic window that encloses acoustic element array 111 and allows ultrasound to pass therethrough, and a frame that holds acoustic element array 111 so as to be rocked.

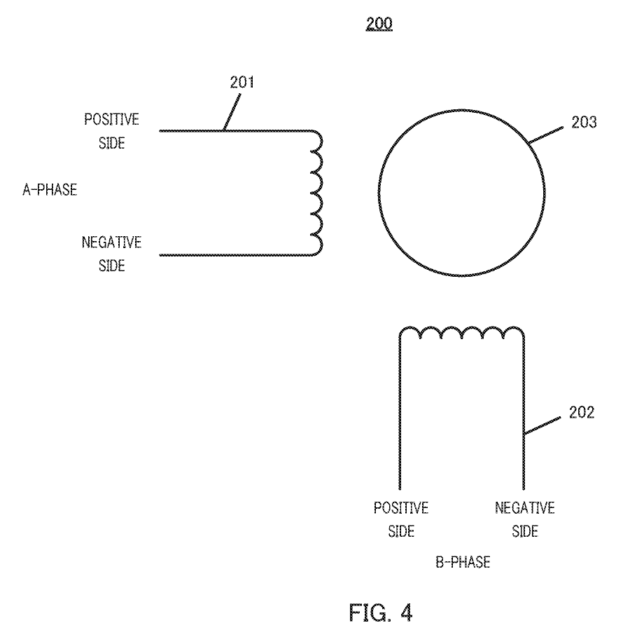

FIG. 4 illustrates an example structure of stepping motor 200 of rocking mechanism 112. As illustrated in FIG. 4, the stepping motor includes two coils 201 and 202, and rotor 203. Two coils 201 and 202 are arranged so as be shifted from each other by an electrical angle of 90°. Accordingly, the directions of the magnetic fields of two coils 201 and 202 with respect to rotor 203 are also shifted from each other by an electrical angle of 90° with respect to the center angle of rotor 203. FIG. 4 illustrates coil 201 as the A-phase side, and coil 202 as the B-phase side.

Rotor 203, which includes a magnet, such as a permanent magnet, is configured to be stabilized at a position corresponding to the magnetic fields of two coils 201 and 202. Accordingly, by supplying alternating current with a phase difference of 90° to two coils 201 and 202, rotor 203 is rotated due to the current phases. Also, by terminating shifts in the current phases at the timing of specific current phases, rotor 203 can be stopped at a position corresponding to the current phases at the moment. Due to this configuration, the rotation of stepping motor 200 is controlled.

As shown in FIG. 3, connector housing 120 includes driving circuit 121, control circuit 122, and connector 123. Driving circuit 121 performs driving control of stepping motor 200 of rocking mechanism 112. Control circuit 122 controls driving circuit 121 on the basis of a command signal from the ultrasound diagnostic apparatus body, for example.

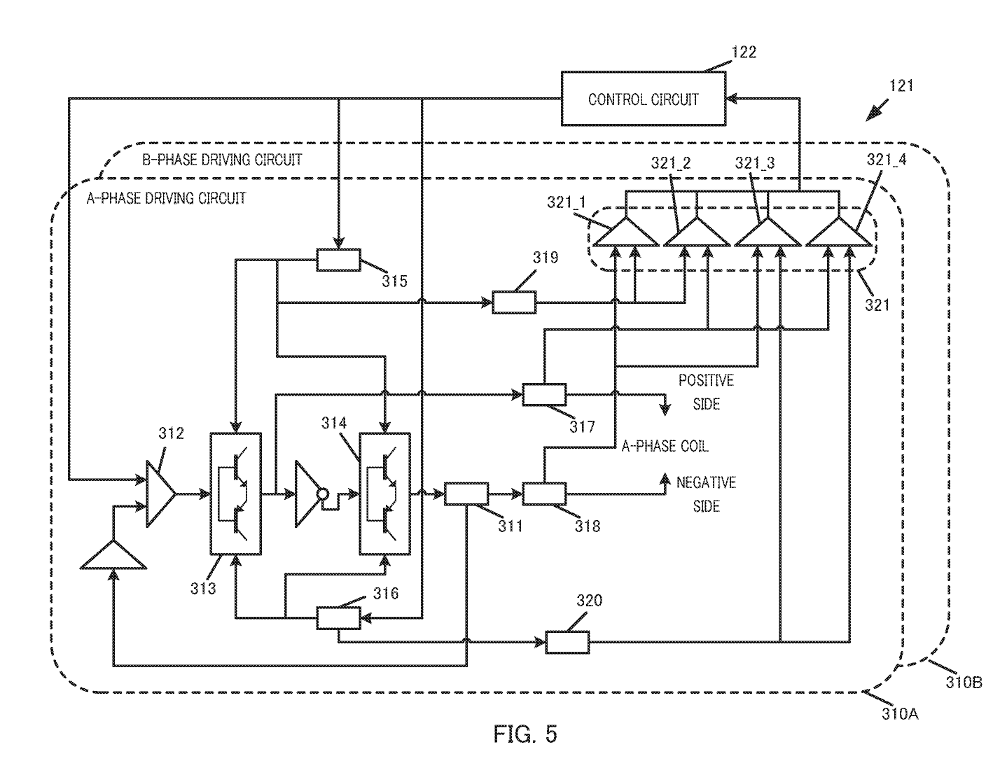

FIG. 5 illustrates an example configuration of driving circuit 121 and control circuit 122. As illustrated in FIG. 5, driving circuit 121 includes A-phase driving circuit 310A and B-phase driving circuit 310B.

Control circuit 122 is an electronic circuit, such as a central processing unit (CPU) or a microprocessing unit (MPU), or is an integrated circuit, such as an application specific integrated circuit (ASIC) or a field programmable gate array (FPGA), for example, and controls driving circuit 121 (A-phase driving circuit 310A and B-phase driving circuit 310B).

As illustrated in FIG. 5, A-phase driving circuit 310A includes current detection section 311, differential amplifier 312, power amplifiers 313 and 314, variable-output switch-mode power supplies 315 and 316, voltage dividers 317 and 318, and comparator 321. The illustration and description of B-phase driving circuit 310B will be omitted since the B-phase driving circuit 310B and A-phase driving circuit 310A have virtually the same configuration.

<Control of A-Phase Driving Circuit 310A>

The outline of the control of A-phase driving circuit 310A by control circuit 122 will be described. For example, when the ultrasound diagnostic apparatus body commands control circuit 122 to rock acoustic element array 111, control circuit 122 generates A-phase phase data (sine wave data) for A-phase driving circuit 310A and B-phase phase data (sine wave data) with a phase difference from the A-phase phase data of 90°, on the basis of the rotation angle (electrical angle) of a motor corresponding to the command. Control circuit 122 then generates an A-phase current command value and a B-phase current command value on the basis of the generated A-phase phase data and B-phase phase data, respectively.

Control circuit 122 inputs the generated A-phase current command value into A-phase driving circuit 310A and the B-phase current command value into B-phase driving circuit 310B. In the following, the operation of A-phase driving circuit 310A, into which the A-phase current command value is input, will be described.

Differential amplifier 312 detects a difference between the input A-phase current command value and a current value (or amplified value thereof) of coil 201 on the A-phase side of stepping motor 200, which is detected by current detection section 311.

Power amplifiers 313 and 314 are analog amplifiers that amplify input current. Differential amplifier 312 and power amplifier 313 constitute a linear amplifier (class-AB amplifier, for example). The output terminal of power amplifier 313 is connected to the input terminal of power amplifier 314 via an inverting circuit, such as an operational amplifier. The inverting circuit and power amplifier 314 constitute a linear amplifier.

Further, the output terminal of power amplifier 313 is connected to a positive side terminal of A-phase coil 201 of stepping motor 200 via voltage divider 317 described hereinafter. In other words, power amplifier 313 is a positive side amplifier of stepping motor 200. Meanwhile, the output terminal of power amplifier 314 is connected to a negative side terminal of coil 201 via voltage divider 318 described hereinafter. Power amplifiers 313 and 314 operate on the basis of the output voltages of variable-output switch-mode power supplies 315 and 316.

Variable-output switch-mode power supplies 315 and 316 are power supplies for stepping motor 200. Variable-output switch-mode power supply 315 is connected to the positive side of coil 201 of stepping motor 200 via power amplifier 313, whereas variable-output switch-mode power supply 316 is connected to the negative side of coil 201 via power amplifier 314. Further, variable-output switch-mode power supply 315 is connected to the high sides (positive sides) of power amplifiers 313 and 314, whereas variable-output switch-mode power supply 316 is connected to the low sides (negative sides) of power amplifiers 313 and 314.

As described above, power amplifiers 313 and 314 share variable-output switch-mode power supply 315 as a high-side power supply, and variable-output switch-mode power supply 316 as a low-side power supply. Due to this configuration, the control of power supplies can be collectively performed by variable-output switch-mode power supply 315 when the driving voltage of stepping motor 200 is positive, and by variable-output switch-mode power supply 316 when the driving voltage is negative. Hereinafter, the output voltage of variable-output switch-mode power supply 315 is denoted by Vₒᵤₜ₊ and the output voltage of variable-output switch-mode power supply 316 is denoted by Vₒᵤₜ₋.

In the embodiment of the present invention, “a positive driving voltage” is not necessarily limited to a case in which a voltage value of the driving voltage is positive, and “a negative driving voltage” is not necessarily limited to a case in which a voltage value of the driving voltage is negative. In the embodiment of the present invention, for example, when a predetermined reference voltage and a driving voltage are compared, a driving voltage higher than the reference voltage is referred to as “a positive driving voltage,” and a driving voltage lower than the reference voltage as “a negative driving voltage.” The predetermined reference voltage is, for example, a specifically set voltage by a designer of ultrasound diagnostic apparatus 1. In addition to the driving voltage, the same also applies to output voltages of variable-output switch-mode power supplies 315 and 316.

Voltage dividers 317 and 318 obtain voltage values of the current paths toward coil 201 and input them into comparator 321. The voltage values of the current paths toward coil 201 herein mean the driving voltages V_{D} of stepping motor 200.

Voltage dividers 319 and 320 obtain output voltages Vₒᵤₜ₊ and Vₒᵤₜ₋ of variable-output switch-mode power supplies 315 and 316, and input them into comparator 321.

Comparator 321 includes a plurality of comparators 321_1 to 321_4. To respective comparators 321_1 to 321_4, voltage values of the current paths toward coil 201 (driving voltages V_{D} of stepping motor 200) obtained by voltage dividers 317 and 318, as well as output voltages Vₒᵤₜ₊ and Vₒᵤₜ₋ of variable-output switch-mode power supplies 315 and 316 are supplied.

Specifically, as illustrates in FIG. 5, the input terminal of comparator 321_1 is connected to the output terminal of voltage divider 318 and the output terminal of variable-output switch-mode power supply 315. As also illustrated in FIG. 5, the input terminal of comparator 321_2 is connected to the output terminal of variable-output switch-mode power supply 315 and the output terminal of voltage divider 317.

Further, as illustrated in FIG. 5, the input terminal of comparator 321_3 is connected to the output terminal of voltage divider 318 and the output terminal of variable-output switch-mode power supply 316. As also illustrated in FIG. 5, the input terminal of comparator 321_4 is connected to the output terminal of voltage divider 317 and the output terminal of variable-output switch-mode power supply 316.

When the driving voltage V_{D} of stepping motor 200 is positive on the basis of output voltages of voltage dividers 317 and 318 as well as variable-output switch-mode power supplies 315 and 316, comparator 321 compares a value in which a predetermined value α is added to the driving voltage V_{D} (V_{D}+α) with an absolute output voltage value Vₒᵤₜ₊ of variable-output switch-mode power supply 315, and outputs the comparison result to control circuit 122. When the driving voltage V_{D} of stepping motor 200 is negative, comparator 321 compares a value in which a predetermined value α is subtracted from the driving voltage V_{D} (V_{D}−α) with the output voltage Vₒᵤₜ₋ of variable-output switch-mode power supply 316, and outputs the comparison result to control circuit 122. Hereinafter, the value in which a predetermined value α is added to the positive driving voltage (V_{D}+α) is referred to as a positive target voltage, and the value in which a predetermined value α is subtracted from the negative voltage V_{D} (V_{D}−α) as a negative target voltage. The positive target voltage (V_{D}+α) and the negative target voltage (V_{D}−α) herein are examples of the first target voltage of the present invention, and the output voltages Vₒᵤₜ₊ and Vₒᵤₜ₋ of variable-output switch-mode power supplies 315 and 316, which comparator 321 compares the positive target voltage (V_{D}+α) and the negative target voltage (V_{D}−α) with, are examples of the second target voltage of the present invention.

Control circuit 122 controls A-phase driving circuit 310A having the above configuration. Specifically, control circuit 122 adjusts the A-phase current command value such that a difference between the A-phase current command value and a current value of coil 201 of stepping motor 200 becomes zero in differential amplifier 312. Due to this, constant current control is performed so that a current of coil 201 always becomes the A-phase current command value.

Control circuit 122 controls variable-output switch-mode power supplies 315 and 316 on the basis of a comparison result by comparator 321 as follows.

(1) When the driving voltage V_{D} is positive, and the output voltage Vₒᵤₜ₊ of variable-output switch-mode power supply 315 is equal to or higher than the positive target voltage (V_{D}+α) as a result of the comparison in comparator 321 (Vₒᵤₜ₊≥V_{D}+α), control circuit 122 reduces the output voltage Vₒᵤₜ₊ of variable-output switch-mode power supply 315 to the positive target voltage (V_{D}+α). Further, when the driving voltage V_{D} is positive, control circuit 122 performs control such that the output voltage Vₒᵤₜ₋ of variable-output switch-mode power supply 316 becomes −α.

(2) When the driving voltage V_{D} is positive, and the output voltage Vₒᵤₜ₊ of variable-output switch-mode power supply 315 is lower than the positive target voltage (V_{D}+α) as a result of the comparison in comparator 321 (Vₒᵤₜ₊<V_{D}+α), control circuit 122 increases the output voltage Vₒᵤₜ₊ of variable-output switch-mode power supply 315 to the positive target voltage (V_{D}+α) or higher so that the output voltage Vₒᵤₜ₊ does not fall below the positive target voltage (V_{D}+α).

(3) When the driving voltage V_{D} is negative, and the output voltage Vₒᵤₜ₋ of variable-output switch-mode power supply 316 is equal to or lower than the negative target voltage (V_{D}−α) as a result of the comparison in comparator 321 (Vₒᵤₜ₋≤V_{D}−α), control circuit 122 increases the output voltage Vₒᵤₜ₋ of variable-output switch-mode power supply 316 to the negative target voltage (V_{D}−α). Further, when the driving voltage V_{D} is negative, control circuit 122 performs control such that the output voltage Vₒᵤₜ₊ of variable-output switch-mode power supply 315 becomes α.

(4) When the driving voltage V_{D} is negative, and the output voltage Vₒᵤₜ₋ of variable-output switch-mode power supply 316 is higher than the negative target voltage (V_{D}−α) as a result of the comparison in comparator 321 (Vₒᵤₜ₋>V_{D}−α), control circuit 122 reduces the output voltage Vₒᵤₜ₋ to the negative target voltage (V_{D}−α) or lower so that the output voltage Vₒᵤₜ₋ does not exceed the target voltage (V_{D}−α).

The above controls (1) to (4) are summarized as follows. When the absolute output voltage values |Vₒᵤₜ₊| and |Vₒᵤₜ₋| of variable-output switch-mode power supplies 315 and 316 become smaller than the absolute target voltage values |V_{D}+α| and |V_{D}−α|, which are set on the basis of the driving voltage V_{D} of stepping motor 200, control circuit 122 performs control such that the absolute output voltage values |Vₒᵤₜ₊| and |Vₒᵤₜ₋| of variable-output switch-mode power supplies 315 and 316 are increased to the absolute target voltage values |V_{D}+α| and |V_{D}−α| or higher.

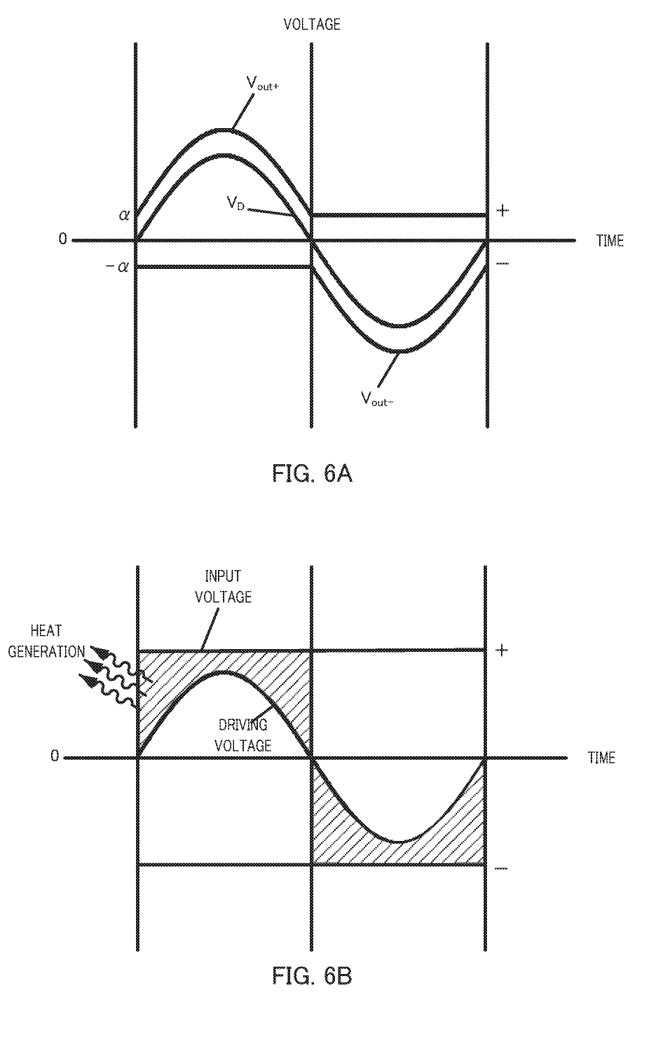

Through such control by control circuit 122, as shown in FIG. 6A, the output voltage Vₒᵤₜ₊ of variable-output switch-mode power supply 315 is controlled to become the positive target voltage (V_{D}+α) (when V_{D}>0). As also shown in FIG. 6A, the output voltage Vₒᵤₜ₋ of variable-output switch-mode power supply 316 is controlled to become the negative target voltage (V_{D}−α) (when V_{D}<0). The predetermined value α may be set to a value of a voltage drop across one diode, for example.

FIG. 6A shows a relationship between the driving voltage V_{D} of stepping motor 200 of ultrasound probe unit 100 according to the embodiment of the present disclosure, and the output voltage Vₒᵤₜ₊ of variable-output switch-mode power supply 315 and the output voltage Vₒᵤₜ₋ of variable-output switch-mode power supply 316.

As described above, the output voltages Vₒᵤₜ₊ and Vₒᵤₜ₋ of variable-output switch-mode power supplies 315 and 316 are input voltages into power amplifiers 313 and 314, and the driving voltage V_{D} of stepping motor 200 is output voltages of power amplifiers 313 and 314. In general, heat generation by power amplifiers 313 and 314 becomes larger as the difference between the input power and the output power of power amplifiers 313 and 314 becomes larger. In ultrasound probe unit 100 of the embodiment of the present disclosure, control circuit 122 performs the voltage control as shown in FIG. 6A, and thus the difference between the input power and the output power of power amplifiers 313 and 314 becomes small. Consequently, heat generation by power amplifiers 313 and 314 can become suppressed.

As a comparative example, FIG. 6B shows a relationship between the input voltage and the output voltage of a power amplifier when the input voltage into the power amplifier is not controlled. As shown in FIG. 6B, in a conventional power amplifier that does not control the input voltage, for example, the difference between the input power and the output power is particularly large, compared to ultrasound probe unit 100 of the embodiment of the present disclosure. Accordingly, ultrasound probe unit 100 according to the embodiment of the present disclosure can significantly reduce heat generation by power amplifiers 313 and 314, compared to the conventional one.

In the foregoing, the outline of the control of A-phase driving circuit 310A by control circuit 122 is described. Since the control of B-phase driving circuit 310B by control circuit 122 is virtually the same as the control of A-phase driving circuit 310A, the description will be omitted.

<Switching Control of Variable-Output Switch-Mode Power Supplies 315 and 316>

Control circuit 122 performs control of variable-output switch-mode power supplies 315 and 316 as follows. In the following, the configuration and the control of variable-output switch-mode power supply 315 will be described, whereas the description of the configuration and the control of variable-output switch-mode power supply 316 will be omitted since they are virtually the same as those of variable-output switch-mode power supply 315.

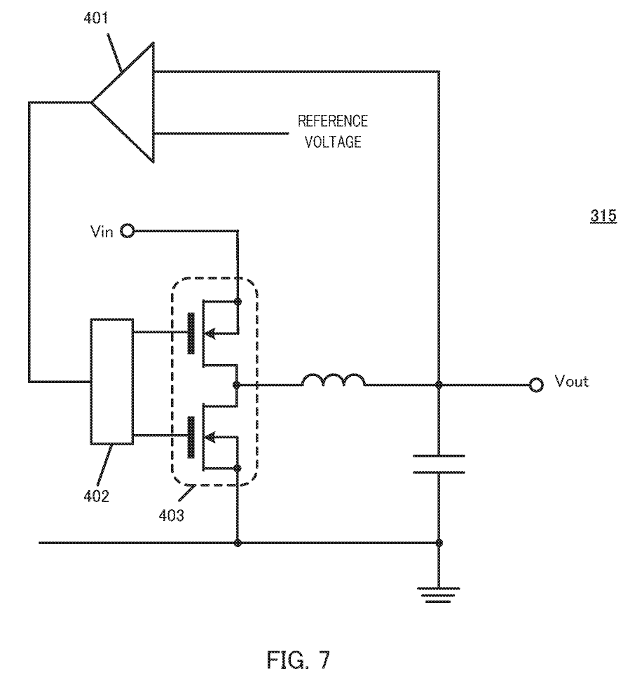

FIG. 7 illustrates a circuit configuration of variable-output switch-mode power supply 315. As illustrated in FIG. 7, variable-output switch-mode power supply 315 includes comparator 401, control circuit 402, and switching element 403.

Comparator 401 compares a predetermined reference voltage with an output feedback voltage, and outputs the comparison result to control circuit 402. The reference voltage input into comparator 401 is the above-described positive target voltage (V_{D}+α). The other input into comparator 401 is the output voltage Vₒᵤₜ₊ of variable-output switch-mode power supply 315. In other words, comparator 401 compares the real-time output voltage Vₒᵤₜ₊ of variable-output switch-mode power supply 315 with the positive target voltage (V_{D}+α).

Control circuit 402 operates under the control by control circuit 122. When the real-time output voltage Vₒᵤₜ₊ of variable-output switch-mode power supply 315 becomes lower than the positive target voltage (V_{D}+α) in a comparison result by comparator 401 (Vₒᵤₜ₊<V_{D}+α), in other words, in the case of the control (2), control circuit 402 performs control such that the output voltage Vₒᵤₜ₊ of variable-output switch-mode power supply 315 does not fall below the positive target voltage (V_{D}+α) by performing frequency-variable pulse frequency modulation (PFM) control (constant off-time), for example, using switching element 403.

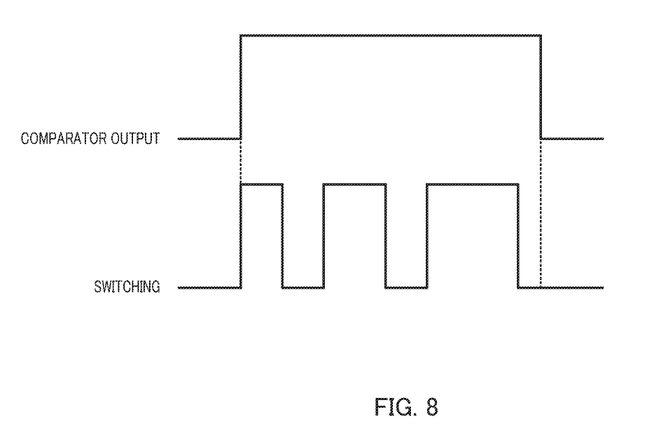

FIG. 8 shows an example signal waveform in PFM control by control circuit 402. As shown in FIG. 8, control circuit 402 changes the switching frequency during the period when the output voltage of comparator 401 is at a high level, i.e., during the period when the real-time output voltage Vₒᵤₜ₊ of variable-output switch-mode power supply 315 becomes lower than the positive target voltage (V_{D}+α). FIG. 8 shows a case in which the switching frequency is changed so that the switching frequency decreases gradually.

FIG. 8 shows a case in which the switching frequency is changed so that the switching frequency decreases gradually when the output voltage of comparator 401 is at a high level. The present disclosure, however, is not limited to this. Alternatively, control circuit 402 that is controlled by control circuit 122 may change the switching frequency so that the switching frequency increases gradually when the output voltage of comparator 401 is at a high level. For example, the switching frequency may also be changed in the opposite directions for every cycle of the driving waveform of stepping motor 200. The switching frequency may be synchronized with the signal transmission cycle of acoustic element array 111 so as not to interfere with the transmission ultrasound, which is transmitted by acoustic element array 111.

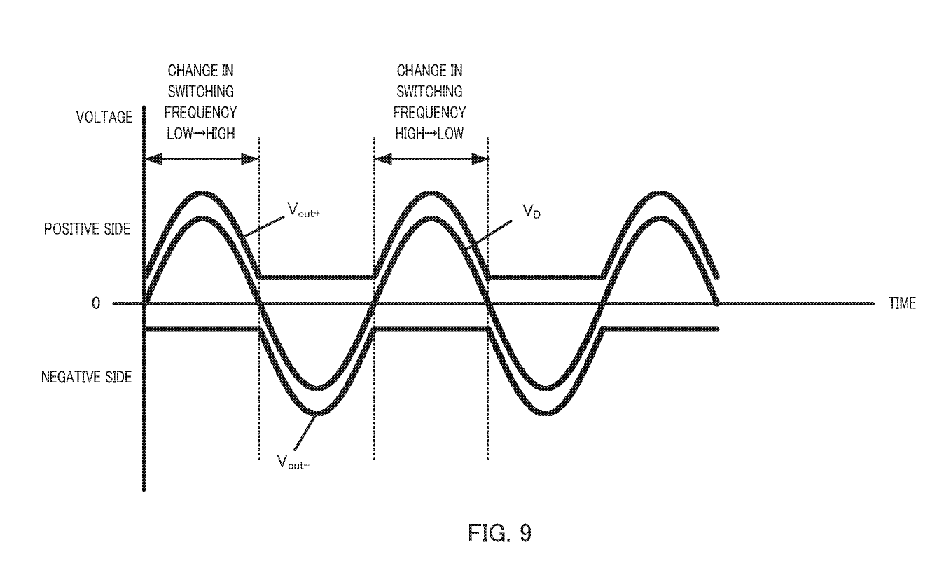

The opposite directions herein specifically mean the following changes. As shown in FIG. 9, for example, control circuit 402 alternately changes the changing direction of the switching frequency by changing the output voltage Vₒᵤₜ₊ of variable-output switch-mode power supply 315 such that the switching frequency increases gradually during the period of the first peak of the driving voltage waveform (sine wave) of stepping motor 200, and the switching frequency decreases gradually during the period of the following peak. FIG. 9 shows an output waveform of variable-output switch-mode power supply 315 when the switching frequency is changed in the opposite directions for every cycle of the driving waveform of stepping motor 200.

Control circuit 402 preferably changes the switching frequency such that the switching frequency is not superimposed on the frequency band of ultrasound probe 110. Specifically, for example, when the frequency band of ultrasound probe 110 is 1 MHz, the switching frequency may be changed in the range of 100 kHz or higher and lower than 1 MHz, for example.

By the control with changing switching frequency as described above, the switching frequency is dispersed, and consequently switching noise arising from variable-output switch-mode power supply 315 (and variable-output switch-mode power supply 316) can be lowered.

<Control of Stepping Motor 200 During High-Speed Rotation>

The control by control circuit 122 during the high-speed rotation of stepping motor 200 will be described. Control circuit 122 monitors the number of rotations of stepping motor 200, and performs the following control when a predetermined number or higher rotations is reached. In the embodiment, the maximum speed of rotation of stepping motor 200 is set to 600 rpm, and the predetermined number of rotations is set to 400 rpm.

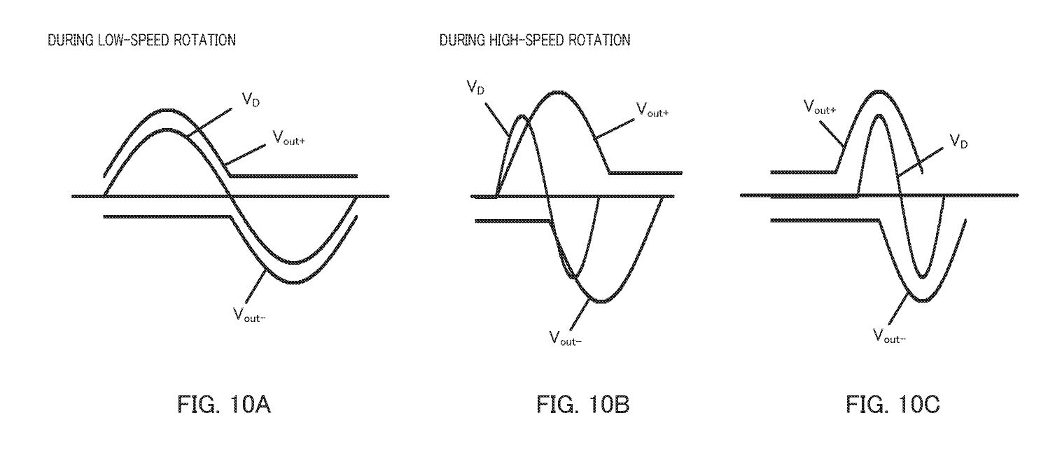

FIG. 10A shows a relationship between the number of rotations of stepping motor 200 and the output waveform of variable-output switch-mode power supply 315. As shown in FIG. 10A, control circuit 122 controls the output voltage Vₒᵤₜ₊ of variable-output switch-mode power supply 315 as described above when stepping motor 200 rotates at a relatively low speed.

When stepping motor 200 rotates at a high speed, however, the change in the driving voltage V_{D} becomes large as shown in FIG. 10B, and thus the control of variable-output switch-mode power supply 315 by control circuit 122 cannot follow the change, and the output voltage Vₒᵤₜ₊ of variable-output switch-mode power supply 315 cannot conform to the target voltage (V_{D}+α) in some cases.

In such a case, control circuit 122 starts the switching control of variable-output switch-mode power supply 315 without waiting a comparison result by comparator 321, as shown in FIG. 10C. Such control is possible since control circuit 122 is notified of the target voltage of the switching control in advance.

Through such control, the switching control of variable-output switch-mode power supplies 315 and 316 can be performed suitably even during the high-speed rotation of stepping motor 200.

<Effects and Advantages>

As described above, ultrasound probe unit 100 according to the embodiment of the present disclosure includes: ultrasound probe 100 including acoustic element array 111 and rocking mechanism 112 having stepping motor 200 that rocks acoustic element array 111 perpendicularly to the scanning direction; connector housing 120 that is connected to ultrasound probe 100 via cable 130, and is connected with the ultrasound diagnostic apparatus body. Connector housing 120 includes driving circuit 121 that drives stepping motor 200, and control circuit 122 that controls driving circuit 121. Driving circuit 121 includes: variable-output switch-mode power supplies 315 and 316; power amplifiers 313 and 314 that output the driving voltage of stepping motor 200 on the basis of the output voltages of variable-output switch-mode power supplies 315 and 316; and comparator 321 that compares the target voltage based on the driving voltage of stepping motor 200 and the output voltages of variable-output switch-mode power supplies 315 and 316. When absolute output voltage values of variable-output switch-mode power supplies 315 and 316 are smaller than absolute target voltage values as a result of the comparison in comparator 321, control circuit 122 performs switching control of variable-output switch-mode power supplies 315 and 316 such that the absolute output voltage values of variable-output switch-mode power supplies 315 and 316 are increased to the absolute target voltage values or higher.

In ultrasound probe unit 100 according to the embodiment of the present disclosure, when absolute output voltage values of variable-output switch-mode power supplies 315 and 316 are equal to or larger than absolute target voltage values as a result of the comparison in comparator 321, control circuit 122 performs switching control of variable-output switch-mode power supplies 315 and 316 such that the absolute output voltage values of variable-output switch-mode power supplies 315 and 316 is decreased to the absolute target voltage values.

Due to this configuration, ultrasound probe unit 100 according to the embodiment of the present disclosure can reduce the difference between the input power and the output power of power amplifiers 313 and 314 that amplify a control current of stepping motor 200, and thus heat generated by power amplifiers 313 and 314 can be reduced. Consequently, the temperature rise in connector housing 120 can be prevented.

In ultrasound probe unit 100 according to the embodiment of the present disclosure, control circuit 122 performs switching control of variable-output switch-mode power supplies 315 and 316 at variable frequencies. Accordingly, the switching frequency is dispersed, and thus switching noise can be lowered. Since control circuit 122 controls the switching frequency such that the switching frequency is not superimposed on the frequency band of the acoustic element array, effects of switching noise superimposed on ultrasonic reception signals, which are transmitted from ultrasound probe 110 to the ultrasound diagnostic apparatus body via connector housing 120, can be lowered.

In ultrasound probe unit 100 according to the embodiment of the present disclosure, when the rotation speed of stepping motor 200 is faster than the predetermined number of rotations (400 rpm), control circuit 122 performs switching control of variable-output switch-mode power supplies 315 and 316 before a comparison result is output from comparator 321. Accordingly, stepping motor 200 can handle the transient response, such as during a shift from low-speed rotation to high-speed rotation.

In ultrasound probe unit 100 according to the embodiment of the present disclosure, variable-output switch-mode power supply 315 is connected to the positive-side terminal of coil 201 of stepping motor 200. The high-side power supply of power amplifier 313, which is a positive-side amplifier, and the high-side power supply of power amplifier 314, which is a negative-side amplifier, share variable-output switch-mode power supply 315. Meanwhile, variable-output switch-mode power supply 316 is connected to the negative-side terminal of coil 201 of stepping motor 200. The low-side power supply of power amplifier 313, which is a positive-side amplifier, and the low-side power supply of power amplifier 314, which is a negative-side amplifier, share variable-output switch-mode power supply 316.

Due to this configuration, the control of power supplies are collectively performed by variable-output switch-mode power supply 315 when the driving voltage of stepping motor 200 is positive, and by variable-output switch-mode power supply 316 when the driving voltage is negative. This can reduce the number of power supplies, compared to a case in which separate power supplies are provided, thereby reducing the circuit scale of driving circuit 121. Connector housing 120 thus can be downsized, and the power consumption by driving circuit 121 can be lowered.

<Modifications>

In the foregoing, the embodiment of the present invention is described with reference to the drawings, but the present invention is not limited to these examples. The technical scope of the present invention encompasses various variations and modifications which a person skilled in the art can conceive within the scope of the Claims. Each feature of the above-described embodiment may be combined optionally without departing from the spirit of the disclosure.

Although a two-phase stepping motor is described as stepping motor 200 of rocking mechanism 112 in the above-described embodiment, the present invention is not limited to this. Rocking mechanism 112 may have a three-phase, five-phase, or other-phase stepping motor, for example. When rocking mechanism 112 has the other-phase stepping motor, driving circuit 122 may be configured so that the number of driving circuits corresponds to the phase number of the stepping motor.

Although comparator 321 sets the positive target voltage by adding a predetermined value α to the driving voltage V_{D} (V_{D}+α) and the negative target voltage by subtracting a predetermined value α from the driving voltage (V_{D}−α) in the above-described embodiment, the present invention is not limited to this. For example, voltage dividers 317 and 318 may be configured to output voltages corresponding to the target voltages (V_{D}+α) and (V_{D}−α) to comparator 321.

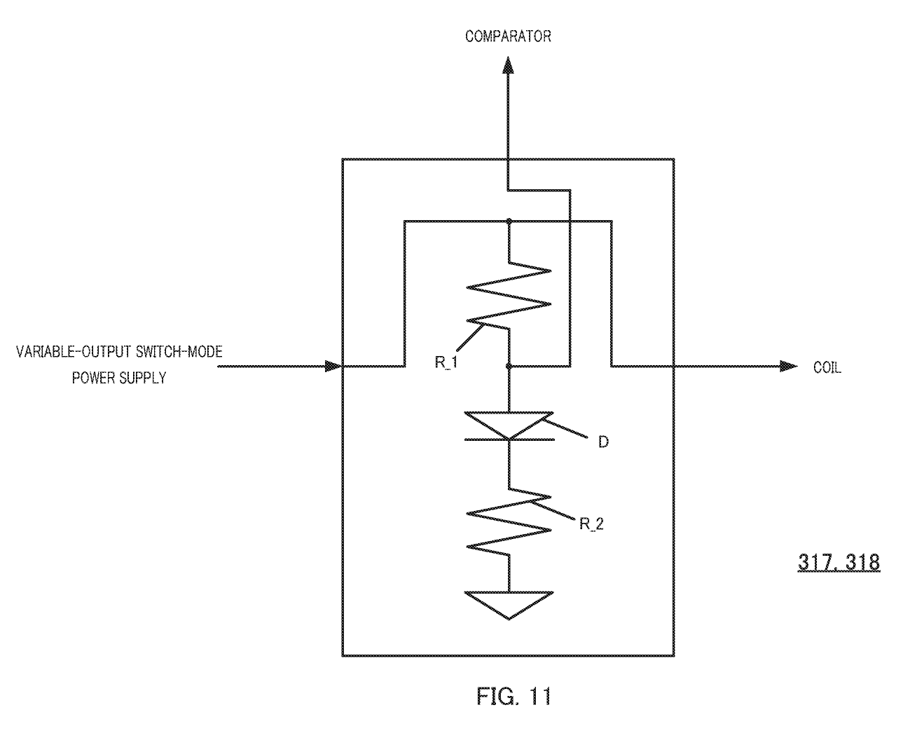

FIG. 11 illustrates an example configuration of voltage dividers 317 and 318 when voltage dividers 317 and 318 output the target voltages (V_{D}+α) and (V_{D}−α) on the basis of the driving voltage V_{D}. As illustrated in FIG. 11, voltage dividers 317 and 318 each include two resistors R_1 and R_2, and diode D provided between the two resistors. One end of resistor R_1 is connected to the input terminal of voltage dividers 317 and 318, and the other end is connected to the output terminal of voltage dividers 317 and 318. The output terminal is connected with the anode of diode D, and the cathode of diode D is connected with one end of resistor R_2. The other end of resistor R_2 is connected with the ground. The input terminal of voltage dividers 317 and 318 is connected to variable-output switch-mode power supplies 315 and 316, respectively, and the output terminal of voltage dividers 317 and 318 is connected to comparator 321. The voltage drop across diode D is set to α×(R_1+R_2)/(R_1−R_2) by taking account of the voltage ratio. In the formula, R_1 is the resistance of resistor R_1, and R_2 is the resistance of resistor R_2.

Due to this configuration, the target voltages can be set to (V_{D}+α) and (V_{D}−α).

In the example illustrated in FIG. 11, voltage dividers 317 and 318 output voltages corresponding to the target voltages (V_{D}+α) and (V_{D}−α) to comparator 321, whereas voltage dividers 319 and 320 output voltages corresponding to the output voltages Vₒᵤₜ₊ and Vₒᵤₜ₋ of variable-output switch-mode power supplies 315 and 316 to comparator 321 in virtually the same manner as in the above-described embodiment.

Alternatively, in the present invention, voltage dividers 319 and 320 may also have a circuit configuration similar to that of FIG. 11, and the output voltages of voltage dividers 317 and 318 may be set to voltages corresponding to (V_{D}+α1) and (V_{D}−α1), and the output voltages of voltage dividers 319 and 320 may be set to voltages corresponding to (Vₒᵤₜ₊+α2) and (Vₒᵤₜ₊−α2). In this case, the comparison similar to that of the above-described embodiment becomes possible by comparing the output voltages of voltage dividers 317 and 318 with the output voltages of voltage dividers 319 and 320 by comparator 321, where α1 is a voltage value obtained from a voltage drop across the diode of voltage dividers 317 and 318, α2 is a voltage value obtained from a voltage drop across the diode of voltage dividers of 319 and 320, and α1−α2=α (when α1>α2). In this configuration, the difference between the voltage drop across the diode based on α1 of voltage dividers 317 and 318, and the voltage drop across the diode based on α2 of voltage dividers 319 and 320 is set to the predetermined value α. Accordingly, the temperature characteristics of the dioses can preferably be compensated for.

Although a case in which driving circuit 122 that controls stepping motor 200 and control circuit 122 are provided inside connector housing 120 of ultrasound probe unit 100 is described in the above-described embodiment, the present invention is not limited to this. For example, a driving circuit that controls a motor and a control circuit may be provided inside an ultrasound probe. In this case, similar to the above-described embodiment, advantages, such as lowered heat generation by a driving circuit (linear amplifier), lowered switching noise against ultrasonic reception signals transmitted from an ultrasound probe to an ultrasound diagnostic apparatus body, and lowered power consumption by a driving circuit, can also be obtained.

Further, a driving circuit and a control circuit, for example, may be provided inside an ultrasound diagnostic apparatus body in the present invention. In this case, since a housing for an ultrasound diagnostic apparatus body is larger than a connector housing, heat generation by the driving circuit rarely causes a problem. Moreover, if a driving circuit and a control circuit are arranged so that the distances between a cable that connects an ultrasound probe and an ultrasound diagnostic apparatus body, and the driving circuit and the control circuit become long, the situation in which switching noise is superimposed on ultrasonic reception signals can be avoided. Further, the power consumption by the driving circuit can be lowered.

### INDUSTRIAL APPLICABILITY

The present invention is suitable for an ultrasound probe unit of an ultrasound diagnostic apparatus that utilizes ultrasound.

Although embodiments of the present invention have been described and illustrated in detail, it is clearly understood that the same is by way of illustration and example only and not limitation, the scope of the present invention should be interpreted by terms of the appended claims.

What is claimed is:

1. An ultrasound probe unit comprising: an ultrasound probe including an acoustic element array and a rocking mechanism having an actuator that moves the acoustic element array in a direction crossing a scanning direction; a driving circuit that drives the actuator; and a control circuit that controls the driving circuit, wherein:
the driving circuit includes: a variable-output switch-mode power supply; a power amplifier into which an output voltage of the variable-output switch-mode power supply is input, and which outputs a driving voltage to the actuator on the basis of the output voltage; and a comparator that compares a first target voltage based on the driving voltage of the actuator with a second target voltage based on the output voltage of the variable-output switch-mode power supply, and
when an absolute value of the second target voltage is smaller than an absolute value of the first target voltage as a result of comparison in the comparator, the control circuit performs switching control of the variable-output switch-mode power supply so as to increase the absolute value of the second target voltage to the absolute value of the first target voltage or larger.

2. The ultrasound probe unit according to claim 1, wherein the control circuit performs constant current control such that an input current into the actuator becomes a predetermined command current value.

3. The ultrasound probe unit according to claim 1, wherein when the absolute value of the second target voltage is equal to or larger than the absolute value of the first target voltage as a result of the comparison in the comparator, the control circuit performs switching control of the variable-output switch-mode power supply so as to reduce the absolute value of the second target voltage to the absolute value of the first target voltage.

4. The ultrasound probe unit according to claim 1, wherein when the control circuit performs switching control of the variable-output switch-mode power supply, the control circuit changes a switching frequency such that the switching frequency decreases gradually.

5. The ultrasound probe unit according to claim 1, wherein when the control circuit performs switching control of the variable-output switch-mode power supply, the control circuit changes a switching frequency such that the switching frequency increases gradually.

6. The ultrasound probe unit according to claim 1, wherein when the control circuit performs switching control of the variable-output switch-mode power supply, the control circuit changes a changing direction of a switching frequency for every cycle of an operation waveform of the actuator.

7. The ultrasound probe unit according to claim 1, wherein when the control circuit performs switching control of the variable-output switch-mode power supply, the control circuit controls a switching frequency such that the switching frequency is synchronized with a signal transmission cycle of the acoustic element array.

8. The ultrasound probe unit according to claim 1, wherein when the control circuit performs switching control of the variable-output switch-mode power supply, the control circuit controls a switching frequency such that the switching frequency is not superimposed on a frequency band of the acoustic element array.

9. The ultrasound probe unit according to claim 1, wherein:
the actuator is a motor, and
when a rotation speed of the motor is faster than a predetermined number of rotations, the control circuit performs switching control of the variable-output switch-mode power supply before a comparison result is output from the comparator.

10. The ultrasound probe unit according to claim 1, wherein:
the power amplifier includes a positive-side power amplifier that supplies a positive current to the actuator and a negative-side power amplifier that supplies a negative current to the actuator; and
the variable-output switch-mode power supply includes a positive-side power supply that supplies power to a high side of the positive-side power amplifier and a high side of the negative-side power amplifier, and a negative-side power supply that supplies power to a low side of the positive-side power amplifier and a low side of the negative-side power amplifier.

11. The ultrasound probe unit according to claim 1, wherein the first target voltage is a voltage which is the driving voltage increased or decreased by a predetermined value.

12. The ultrasound probe unit according to claim 1, wherein the second target voltage is a voltage which is the output voltage increased or decreased by a predetermined value.

13. The ultrasound probe unit according to claim 1, further comprising a connector housing that is connected to the ultrasound probe via a cable, and that is connected with an ultrasound diagnostic apparatus body, wherein the driving circuit and the control circuit are provided inside the connector housing.

14. An ultrasound diagnostic apparatus comprising the ultrasound probe unit according to claim 13, and the ultrasound diagnostic apparatus body, wherein
the ultrasound diagnostic apparatus body causes the ultrasound probe to transmit an ultrasonic transmission signal to a test object, and generates an ultrasound image on the basis of an ultrasonic reception signal generated by the ultrasound probe that has received a reflected wave from the test object.

15. An ultrasound diagnostic apparatus comprising an ultrasound probe unit and an ultrasound diagnostic apparatus body that causes the ultrasound probe unit to transmit an ultrasonic transmission signal to a test object, and generates an ultrasound image on the basis of an ultrasonic reception signal generated by the ultrasound probe unit that has received a reflected wave from the test object, wherein:
the ultrasound probe unit includes: an ultrasound probe including an acoustic element array and a rocking mechanism having an actuator that rocks the acoustic element array perpendicularly to a scanning direction; and a connector housing that is connected to the ultrasound probe via a cable, and that is connected with the ultrasound diagnostic apparatus body;
the ultrasound diagnostic apparatus body includes a driving circuit that drives the actuator and a control circuit that controls the driving circuit;
the driving circuit includes: a variable-output switch-mode power supply; a power amplifier into which an output voltage of the variable-output switch-mode power supply is input, and which outputs a driving voltage to the actuator on the basis of the output voltage; and a comparator that compares a first target voltage based on the driving voltage of the actuator with a second target voltage based on the output voltage of the variable-output switch-mode power supply; and
when an absolute value of the second target voltage is smaller than an absolute value of the first target voltage as a result of comparison in the comparator, the control circuit performs switching control of the variable-output switch-mode power supply so as to increase the absolute value of the second target voltage to the absolute value of the first target voltage or larger.
